(19) United States
(12) Patent Application Publication
(10) Pub. No.: US 2024/0180492 A1
(43) Pub. Date: Jun. 6, 2024

(54) Signal Transmitting Element

(71) Applicant: METAL INDUSTRIES RESEARCH & DEVELOPMENT CENTRE, Kaohsiung (TW)

(72) Inventors: Chun-Chieh Tseng, Kaohsiung (TW); Chun-Ming Chen, Kaohsiung (TW); Tung-Lin Tsai, Tainan (TW); Yen-Hao Chang, Yuanlin City (TW); Shu-Hung Huang, Kaohsiung (TW); Sheng-Hua Wu, Kaohsiung (TW); Yen-Hsin Kuo, Kaohsiung (TW); Ping-Ruey Chou, New Taipei (TW)

(21) Appl. No.: 18/072,941

(22) Filed: Dec. 1, 2022

(51) Int. Cl. A61B 5/00; A61B 5/026; A61B 5/07

(52) U.S. Cl. CPC A61B 5/6876; A61B 5/026; A61B 5/07; C25D 7/0607

(57) ABSTRACT

A signal transmitting element is used to solve the problem that an additional surgery is required to remove the conventional vascular monitoring element after completing the detecting task. The signal transmitting element comprises a body made of a specific biodegradable material. The body includes a signal sensing portion including a structure configured to sense a blood flow information of a blood vessel surrounded and contacted by the body, thereby generating a blood vessel signal; and a signal transmitting portion coupled with the signal sensing portion for receiving the blood vessel signal and including a specific structure configured to convert the blood vessel signal into a transmission signal.

### BACKGROUND OF THE INVENTION

### 1. Field of the Invention

The present invention relates to a signal transmitting element and, more particularly, to a signal transmitting element that can be degraded in the human body.

### 2. Description of the Related Art

In the conventional clinical technology of vascular monitoring, a probe is used to proceed with automatic timing monitoring of thrombus in a blood vessel after surgery, with the probe being an invasive type and partially exposed outside of the human body of the monitored person, such as Doppler Blood Flow Monitor and corresponding Cool-Swartz Doppler Probe produced by Cook Medical. A portion of the probe is wound around the blood vessel to be monitored. Another portion of the probe extends beyond the human body to form an external connecting section connected with the monitor. The monitor transmits supersonic waves via the probe to obtain the blood flow information of the surrounded blood vessel. However, the external connecting section outside of the human body is apt to be contaminated and fall and tends to be pulled, causing injury to the blood vessel. Furthermore, an additional surgery is required to remove the probe when monitoring is no longer required, such that the patient would be injured again. Furthermore, the blood vessel could be pulled and injured during the surgery.

Thus, improvement to the conventional signal transmitting element is necessary.

### SUMMARY OF THE INVENTION

To solve the above problem, an objective of the present invention is to provide a signal transmitting element which can continuously monitor the target blood vessel of a patient in a specific period of time after the surgery and which can be degraded in the patient's body after the specific period of time without additional surgery, thereby avoiding the risks caused by the additional surgery.

Another objective of the present invention is to provide a signal transmitting element which can enhance the strength of the signal indicative of the blood flow information.

A further objective of the present invention is to provide a signal transmitting element which can enhance the strength of the signal of a specific frequency according to need or increase the breadth of the signal transmission frequency.

Still another objective of the present invention is to provide a signal transmitting element which can obtain a signal transmitting section with a low resistance and a high adhesion.

When the terms “front”, “rear”, “left”, “right”, “up”, “down”, “top”, “bottom”, “inner”, “outer”, “side”, and similar terms are used herein, it should be understood that these terms have reference only to the structure shown in the drawings as it would appear to a person viewing the drawings and are utilized only to facilitate describing the invention, rather than restricting the invention.

As used herein, the term “a” or “an” for describing the number of the elements and members of the present invention is used for convenience, provides the general meaning of the scope of the present invention, and should be interpreted to include one or at least one. Furthermore, unless explicitly indicated otherwise, the concept of a single component also includes the case of plural components.

As used herein, the term “connection”, “engagement”, “combination”, “assembly”, “installation”, “disposition”, or similar terms is used to include separation of connected members without destroying the members after connection or inseparable connection of the members after connection. A person having ordinary skill in the art would be able to select according to desired demands in the material or assembly of the members to be connected.

As used herein, the term “coupling” is used to include direct or indirect connection through electrical connection and/or signal, which can be selected by one having ordinary skill in the art according to needs.

The definitions of measurements of sizes used herein are based on FIG. 1 of the present invention, wherein the term “length” is defined by the extending direction of the length (such as symbol Ln) of the head portion of the body, wherein the term “width” is defined by the extending direction of the width (such as symbol W₁₁ or W₁₂) of the head portion or the extension portion of the body, and wherein the term “thickness” is defined by the extending direction of the thickness (such as symbol T₁₁ or T₁₂) of the head portion or the extension portion of the body. Furthermore, the adjustment interval of the size in these measurements may be 0.1 0.1 μm.

As used herein, the adjustment interval of the “working frequency” may be 1 MHz, the adjustment interval of the “plating temperature” may be 0.1° C., the adjustment interval of “time” may be 1 second, and the adjustment interval of “stirring speed” may be 1 rpm.

A signal transmitting element according to the present invention comprises a body made of a specific biodegradable material. The body includes a signal sensing portion including a structure configured to sense a blood flow information of a blood vessel surrounded and contacted by the body, thereby generating a blood vessel signal; and a signal transmitting portion coupled with the signal sensing portion for receiving the blood vessel signal and including a specific structure configured to convert the blood vessel signal into a transmission signal.

Thus, in the signal transmitting element according to the present invention, by the signal transmitting portion and the signal sensing portion (in the laminated structure) which respectively senses and transmits the corresponding blood vessel signal, continuous monitoring of the target blood vessel of a patient within a specific period of time after surgery can be achieved. Furthermore, the signal transmitting element can be degraded in the patient's body without additional surgery for removal, avoiding the risks of the additional surgery.

In an example, the body is in the form of a strip and includes a head portion and an extension portion extending outwards from the head portion. The extending portion completely or partially surrounds the blood vessel. The signal transmitting portion is disposed in the head portion. The signal sensing portion is disposed in the extension portion. Therefore, by the overall disposition, the body can be easily disposed in the human body of a person to be monitored, thereby achieving monitoring of the target blood vessel.

In an example, the extension portion includes a plurality of protruding structures protruding from a surface thereof, such that when the extension portion surrounds the blood vessel, all or a portion of the plurality of protruding structures contacts with the blood vessel. Therefore, by the disposition of the plurality of protruding structures, the strength of the signal of the blood flow information detected by signal sensing portion can be enhanced.

In an example, the body includes at least one metal structure. Each of the at least one metal structure is made of a biodegradable metal. Each of the at least one metal structure includes a signal transmitting section and a signal sensing section. The signal transmitting section is located in the head portion of the body to form the signal transmitting portion. The signal sensing section is located in the extension portion of the body to form the signal sensing portion. The signal transmitting section has a working frequency used for transmitting the blood flow information sensed by the signal sensing section. Therefore, sensing of the blood flow information and transmission of the blood flow information can be achieved by the signal transmitting section and the signal sensing section of the metal structure.

In an example, the at least one metal structure includes a plurality of metal structures. The working frequency of one of the plurality of metal structures is identical to or different from that of another of the plurality of metal structures. Therefore, by providing the signal transmitting sections of the plurality of metal structures with the same working frequency, the signal transmission strength of the working frequency can be enhanced. Alternatively, by providing the signal transmitting sections of the plurality of metal structures with different working frequencies, the frequency breadth of the signal transmission can be increased.

In an example, the working frequency is between 350 MHz and 450 MHz. Therefore, the signal transmission with a stable signal quality can be achieved by the working frequency.

In an example, the working frequency is between 401 MHz and 406 MHz. Therefore, the signal transmission with a stable signal quality can be achieved by the working frequency.

In an example, the material of each of the at least one metal structure is at least selected from magnesium or magnesium alloy. Therefore, by selecting the material of the metal structure, the metal structure can be made of a biodegradable material, and sensing and transmission of the blood flow information of the target blood vessel can be achieved.

In an example, the signal transmitting element further comprises a gold-plated layer coated on a surface of at least one of the at least one metal structure. Therefore, the gold-plated layer can achieve the effects of reducing the reflection loss, having a lower impedance, increasing the radiation efficiency, and reducing the impedance loss.

In an example, the gold-plated layer has a thickness between 0.01 μm and 10 μm. Therefore, the gold-plated layer can achieve the effects of reducing the reflection loss, having a lower impedance, increasing the radiation efficiency, and reducing the impedance loss.

In an example, the gold-plated layer is formed under a condition including a plating temperature of 15° C.-35° C., a pretreatment time of 60-150 seconds, a plating time of 45-75 seconds, and a stirring speed of 100-300 rpm. Therefore, a signal transmitting section with a low resistance and a high adhesion can be obtained under the condition.

In an example, the gold-plated layer is formed under a condition including a plating temperature of 25° C., a pretreatment time of 120 seconds, a plating time of 60 seconds, and a stirring speed of 150 rpm. Therefore, a signal transmitting section with a low resistance and a high adhesion can be obtained under the condition.

In an example, the body includes a first protective layer and a second protective layer. The at least one metal layer is enveloped between the first protective layer and the second protective layer. The plurality of protruding structures is made of a material including at least polycaprolactone. The first protective layer and the second protective layer are made of a material selected from at least one of polyhydroxybutyrate and polyhydroxyvalerate. Therefore, by enveloping the metal layer between the first protective layer and the second protective layer, the metal layer is less likely to be interfered by the environment of the human body. Furthermore, the body can be made of a biodegradable material by selecting the materials of the first and second protective layers and the protruding structures.

In an example, the at least one metal structure includes a plurality of metal structures. Each two adjacent metal structures have an insulating layer disposed therebetween. The insulating layer is made of a material including at least poly-L-lactic acid. Therefore, by the design of the insulating layer, signal interference between two adjacent metal structures which are electrically insulated can be avoided.

In an example, the head portion has a length between 5 mm and 35 mm, a width between 5 mm and 35 mm, and a thickness between 50 μm and 350 μm. The extension portion has a width between 2 mm and 15 mm and a thickness between 50 μm and 350 μm. Therefore, by the arrangement of the above sizes, the signal transmitting element can be easily disposed in the human body of the person to be detected.

### BRIEF DESCRIPTION OF THE DRAWINGS

FIG. 1 is a schematic perspective view of a signal transmitting element of a preferred embodiment according to the present invention.

FIG. 2 is a schematic view illustrating the use state of the signal transmitting element of FIG. 1.

FIG. 3 is a schematic view illustrating an example of a laminated structure contained in a body of the signal transmitting element according to the present invention.

FIG. 4 is a schematic view illustrating another example of the laminated structure contained in the body of the signal transmitting element according to the present invention.

FIG. 5 is a schematic view illustrating an antenna structure in a signal transmitting section of a metal structure according to the present invention.

### DETAILED DESCRIPTION OF THE INVENTION

The present invention will become clearer in light of the following detailed description of illustrative embodiments of this invention described in connection with the drawings. Furthermore, the elements designated by the same reference numeral in various figures will be deemed as identical, and the description thereof will be omitted.

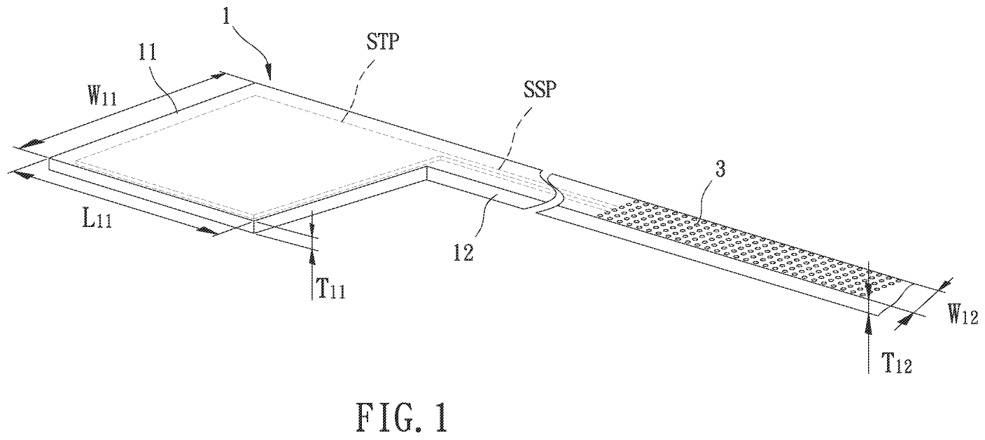

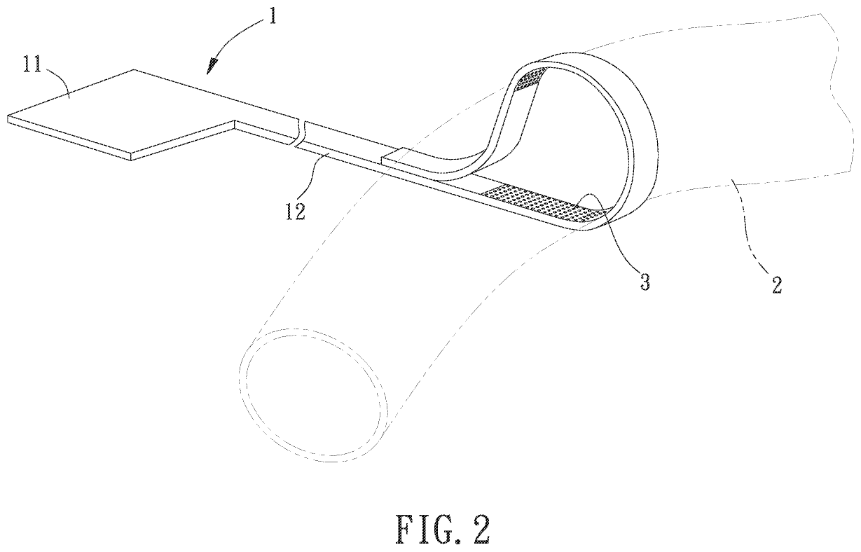

With reference to FIGS. 1 and 2 respectively showing a signal transmitting element and the use state of a preferred embodiment according to the present invention, the signal transmitting element includes a body 1 preferably in the form of a strip which can be used to be partially wound around a blood vessel 2.

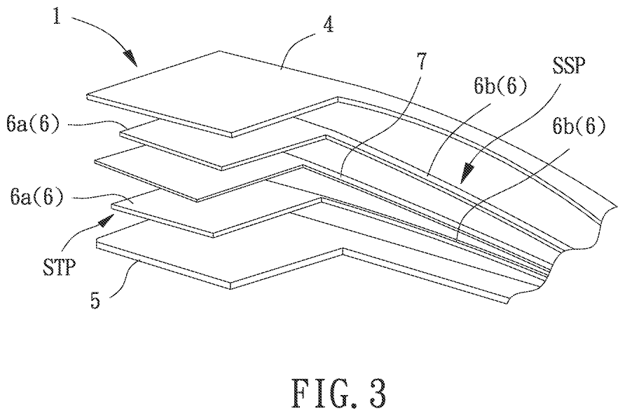

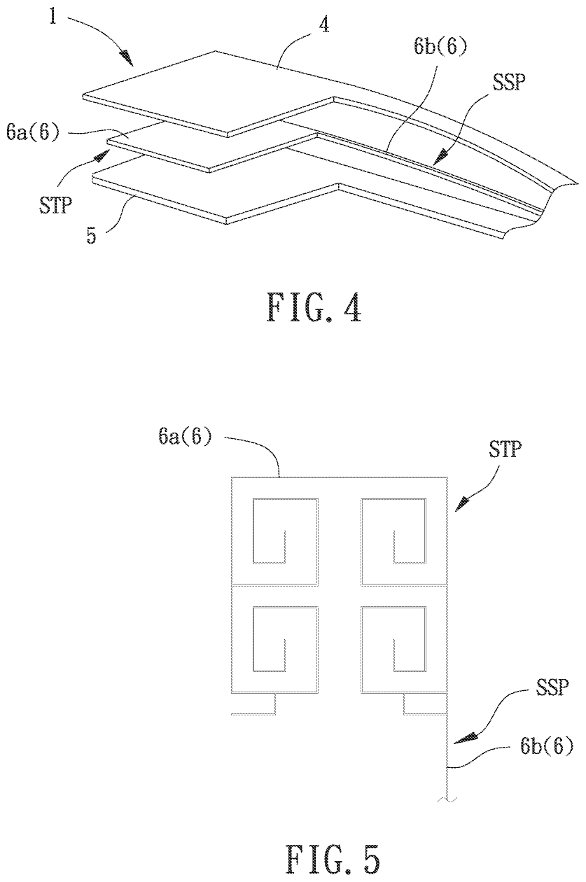

Particularly, the body 1 is made of a biodegradable material and is preferably formed of a laminated structure, as shown in FIGS. 3 and 4. Furthermore, the body 1, particularly at least one layer of the laminated structure, includes a signal transmitting portion STP and a signal sensing portion SSP. The signal transmitting portion STP is used to convert a received blood vessel signal into a transmission signal, and the transmission signal is to be transmitted to a signal identification equipment (not shown), particularly a signal identification equipment disposed outside a human body. The signal identification equipment (such as a radio frequency identification equipment) can receive and analyze the transmission signal to obtain the corresponding blood flow information, which can be used to further assess whether the blood vessel is heathy. The signal sensing portion SSP is used to sense a blood flow information of the blood vessel 2 and converts the blood flow information into the blood vessel signal. Through the coupling disposition (particularly direct connection) between the signal sensing portion SSP and the signal transmitting portion STP, the signal sensing portion SSP transmits the blood vessel signal to the signal transmitting portion STP. Particularly, with the body 1 surrounding the blood vessel 2, the signal sensing portion SSP correspondingly surrounds the blood vessel 2. Therefore, by the signal transmitting portion STP and the signal sensing portion SSP which are made of the biodegradable material, monitoring of a target blood vessel 2 of a patient within a specific period of time after surgery can be achieved. Furthermore, after the specific period of time, the signal transmitting element according to the present invention can be degraded in the patient's body without additional surgery for removal, avoiding the risks of the additional surgery.

Specifically, the body 1 includes a head portion 11 and an extension portion 12 extending outwards from the head portion 11. The extending portion 12 completely or partially surrounds the blood vessel 2. Particularly, when the extension portion 12 surrounds the blood vessel 2, the extension portion 12 forms an annular loop in which a free end of the extension portion 12 may be fixed to a proper portion of the extension portion 12, and the corresponding fixing method can be selected according to the practical situation. The present invention is not limited in this regard. Preferably, the signal transmitting portion STP is disposed in the head portion 11, and the signal sensing portion SSP is disposed in the extension portion 12. Preferably, the head portion 11 has a length L ii between 5 mm and 35 mm, a width W₁₁ between 5 mm and 35 mm, and a thickness T₁₁ between 50 μm and 350 μm. The extension portion 12 has a width W₁₂ between 2 mm and 15 mm and a thickness T₁₂ between 50 μm and 350 μm.

More preferably, the extension portion 12 includes a plurality of protruding structures 3 protruding from a surface thereof. Particularly, the plurality of protruding structures 3 is located corresponding to the position of the blood vessel 2, such that when the extension portion 12 surrounds the blood vessel 2, all or a portion of the plurality of protruding structures 3 contacts with the blood vessel 2. Specifically, when the extension portion 12 surrounds the blood vessel 2, a portion of the extension portion 12 surrounding the blood vessel 2 is in contact with the blood vessel 2, and all or a portion of the plurality of protruding structures 3 contacts with the blood vessel 2. Particularly, the plurality of protruding structures 3 contacting with the blood vessel 2 is in closer contact with the blood vessel 2 than the extension portion 12. Therefore, by the contact between the plurality of protruding structures 3 and the blood vessel 2, the signal strength of the signal sensing portion SSP sensing the blood flow information can be enhanced. Optionally, the protruding structure 3 may, but not limited to, be cylindrical, semi-cylindrical, semi-elliptic, trapezoidal. Preferably, the protruding structure 3 has a length between 0.1 μm and 100 μm, a width between 0.1 μm and 100 μm, and a height between 0.1 μm and 100 μm.

With reference to FIG. 3 illustrating disposition of the laminated structure of the signal transmitting element according to the present invention, the body 1 (the plural layers made of the biodegradable material) includes a first protective layer 4 a second protective layer 5, and at least one metal structure 6. Each of the at least one metal structure 6 may, for example, form a layer enveloped between the first protective layer 4 and the second protective layer 5. It should be noted that the number of the at least one metal structure 6 may be one or plural. In the case that there are plural metal structures 6, any two metal structures 6 are stacked, and two adjacent metal structures 6 have an insulating layer 7 disposed therebetween to electrically insulate the two adjacent metal structures 6, such that signal interference between two adjacent metal structures 6 can be avoided. Preferably, as shown in FIG. 3, the number of the at least one metal structure 6 is two, and an insulating layer 7 is disposed between the two adjacent metal structures 6. In another example, as shown in FIG. 4, the number of the at least one metal structure 6 is one. Preferably, each of the first protective layer 4 and the second protective layer 5 has a thickness not greater than 100 μm. Preferably, the thickness of the metal structure 6 is between 10 μm and 100 μm. Preferably, the thickness of the insulating layer 7 is not greater than 100 μm.

Specifically, the first protective layer 4, the second protective layer 5, each of the at least one metal structure 6 and/or the insulating layer 7 are disposed corresponding to the head portion 11 and the extension portion 12. Particularly, the sizes and shapes of the first protective layer 4 and the second protective layer 5 correspond to the body 1. Each of the at least one metal structure 6 is in the form of a layer or sheet. The shape of each of at least one metal structure 6 corresponds to the first protective layer 4 and the second protective layer 5. Furthermore, the size of each of the at least one metal structure 6 is smaller than the first protective layer 4 and the second protective layer 5 to have the metal structure 6 enveloped between the first protective layer 4 and the second protective layer 5. The shape of the insulating layer 7 corresponds to the associated two adjacent metal structures 6. The size of the insulating layer 7 is not smaller than the associated two adjacent metal structures 6 and not greater than the first and second protective layers 4 and 5 to thereby insulate the associated two adjacent metal structures 6. Furthermore, the insulating layer 7 is enveloped between the first protective layer 4 and the second protective layer 5. Furthermore, the each of the at least one metal structure 6 has a signal transmitting section 6a and a signal sensing section 6b. The signal transmitting section 6a is located in the head portion 11 of the body 1 to form the signal transmitting portion STP. The signal sensing section 6b is located in the extension portion 12 of the body 1 to form the signal sensing portion SSP. Namely, the body 1 includes the at least one metal structure 6, the signal transmitting portion STP is formed by the signal transmitting section 6a, and the signal sensing portion SSP is formed by the signal sensing section 6b. Preferably, the signal transmitting section 6a has a length between 1 mm and 35 mm and a width between 1 mm and 35 mm. More preferably, the signal transmitting section 6a has a length of 10 mm and a width of 10 mm.

Specifically, the signal sensing section 6b may be in the form of a single section (but plural sections are also acceptable) distributed in an area of the body 1 surrounding the blood vessel 2. The signal transmitting section 6a is connected with the signal sensing section 6b to receive the signal (particularly the blood flow information of the blood vessel 2) sensed by the signal sensing section 6b. Furthermore, the signal transmitting section 6a has a working frequency/transmission frequency for transmitting the signal sensed by the signal sensing section 6b. Particularly, as shown in FIG. 5, the signal transmitting section 6a is an antenna structure which is preferably in the form of a flat antenna/planar antenna. The working frequency may be decided by the antenna pattern of the signal transmitting section 6a. Namely, given the same material and size, different working frequencies can be obtained through different antenna patterns. The antenna pattern may also be comprised of at least one of a square loop, a circular loop, a triangular loop, a non-symmetric loop, and other patterns. Preferably, the working frequency is between 350 MHz and 450 MHz. More preferably, the working frequency is between 401 MHz and 406 MHz. Therefore, in the range of the working frequency, the signal transmitting section 6a can proceed with signal transmission with a stable signal quality.

It should be noted that in a case that the body 1 has a plurality of metal structures 6, the working frequency of one of the plurality of metal structures 6 is identical to or different from that of another of the plurality of metal structures 6. Specifically, in an example having at least three metal structures 6, the working frequency may be totally the same, totally different, or partially the same. The term “partially the same” refers to the working frequency of at least two metal structures 6 is different from the working frequency of other metal structure(s) 6. In an example having only two metal structures 6, the working frequencies of the two metal structures 6 are the same or different. Given the same working frequency, the signal transmission strength at the working frequency can be enhanced, which is particularly suitable for a signal transmitting element disposed in a deeper place of a human body. Given different working frequencies, the frequency breadth of the signal transmission can be increased. Therefore, the working frequencies of the metal structures 6 may be determined to be the same or different according to the environmental status of the signal transmitting element according to the present invention in the human body, thereby enhancing the quality of signal transmission.

Specifically, the protruding structures 3, the first protective layer 4, the second protective layer 5, the metal structure 6, and the insulating layer 7 are made of biodegradable materials, particularly the materials approved by U.S. Food and Drug Administration (FDA). Preferably, the degradation time of the biodegradable material in the human body is about six months. The protruding structures 3 is made of a material including at least polycaprolactone (PCL). The first protective layer 4 and the second protective layer 5 are made of a material selected from at least one of polyhydroxybutyrate (PHB) and polyhydroxyvalerate (PHV). The metal structure 6 is made of a biodegradable metal which is at least selected from magnesium or magnesium alloy. Particularly, the metal structure 6 is made of magnesium wires or magnesium alloy wires. Optionally, to enhance the transmission of the signal sensed by the metal structure 6, a surface treated layer (not shown) may be formed on a surface of the metal structure 6 to reduce the resistance of the metal structure 6. Furthermore, the surface treated layer includes materials or structure that will not affect the interior material degradation of the metal structure 6. Preferably, the surface treated layer is a gold-plated layer coated on the surface of the metal structure 6, such as electroplating gold on surfaces of the magnesium wires or magnesium alloy wires to form gold-plated magnesium wires or gold-plated magnesium alloy wires. Furthermore, by arranging the gold-plated layer in a certain thickness range, the interior material of the metal structure 6 may be degraded and absorbed by the human body. The thickness of the gold-plated layer is between 0.01 μm and 10 μm. More preferably, the insulating layer 7 is made of a material including at least poly-L-lactic acid (PLLA).

It should be noted that in the example of the metal structure 6 made of magnesium wires and the surface treated layer in the form of a gold-plated layer, comparing the gold-plated magnesium wires with the magnesium wires before plating, the characteristics of the return loss and the impedance parameter are better. The lower the return loss, the lower return signal during transmission. The impedance parameter is used to observe the matching condition of the antenna at each frequency band. When the resistance and the reactance are higher at the specific frequency band, the antenna will have stronger inductivity and stronger capacitance at the specific frequency band. Thus, the antenna is more difficult to match with the specific frequency breadth, and noise signals and resonance tend to occur in actual operation. In this case, reflection of the incident waves of the transmission signal will occur, resulting in loss of signal. Particularly, in an example of the gold-plated layer with a thickness between 0.01 μm and 10 μm, the overall resistance of the metal structure 6 may be controlled to be in a range between 0.1 ohm and 0.3 ohm, achieving a better signal transmission effect.

Furthermore, in an experimental simulation of the antenna radiation efficiency of the magnesium wires before and after plating, the radiation efficiency in the Z-axis of the magnesium wires is improved after plating, and the torture and unsaturation of the antenna radiation pattern of the magnesium wires before plating are improved. The corresponding antenna radiation frequency can be calculated by equation (1), and the corresponding impedance loss can be calculated by equation (2). Through derivation of the above equations, an increase in the electrical conductivity of material may increase the radiation efficiency and reduce the impedance loss, which can be used as a reference for the signal sensing characteristics after implanting the transducer according to the present invention into the human body.

The equation of radio frequency is as follows:

$${η}_{radiation} \approx {[ 1 + \frac{27}{64} {c}_{0} \sqrt{\frac{π}{{μ}_{0}}} {( {cr}_{0} \sqrt{σ {f}_{0}^{3}} )}^{- 1} coth ( \frac{h}{2 δ} ) ]}^{- 1} \qquad ( 1 )$$

wherein η_{radiation} is the radiation frequency, c₀ is the speed of light, μ₀ is the space permeability, c is the width of antenna, r₀ is the radius of the radiation pattern, σ is the electrical conductivity of material, f₀ is the working frequency, h is the antenna thickness, and δ is the dielectric constant.

The equation of impedance loss is as follows:

$${R}_{loss} \approx \frac{π {r}_{0}}{σ {c}_{eff} δ} coth ( \frac{h}{2 δ} ) \qquad ( 2 )$$

wherein Rₗₒₛₛ is the impedance loss, r₀ is the radius of the radiation pattern, σ is the electrical conductivity of material, c_{eff} is half of the antenna width, h is the antenna thickness, and δ is the dielectric constant.

It should be noted that the above plating process of the magnesium wires is carried out under the specific plating temperature, pretreatment time, plating time, and stirring speed, and preferably obtains the results of a lower impedance, a metal structure which is less likely to peel, and a higher signal-to-noise ratio. Thus, the present invention provides a better plating method carried out at a plating temperature of 15° C.-35° C., a pretreatment time of 60-150 seconds, a plating time of 45-75 seconds, and a stirring speed of 100-300 rpm, which can obtain a product with a resistance not greater than 0.3 ohm, and the actual plating damaged area is smaller than 5% after the adhesion test. Namely, the signal transmitting section 6a with a low resistance and a high adhesion can be obtained under the above specific plating condition.

More preferably, the plating temperature is 25° C., the pretreatment time is 120 seconds, the plating time is 60 seconds, and the stirring speed of 150 rpm. The following Table I shows the experimental result of each parameter combination in treatment.

TABLE 1
related parameters combinations and results of plating process

| | Control parameter | | | | | | |
|---|---|---|---|---|---|---|---|
| | Plating | Pretreatment | Plating | Stirring | Experimental results | | |
| Factor | temperature | time | time | speed | Resistance | Adhesion | S/N |
| combination | (° C.) | (sec) | (sec) | (rpm) | (Ω) | grade | ratio |
| 1 | 0 | 120 | 30 | 50 | 0.2 | 4B | 13.98 |
| 2 | 0 | 180 | 60 | 150 | 0.3 | 3B | 10.46 |
| 3 | 0 | 240 | 120 | 270 | 0.2 | 4B | 13.98 |
| 4 | 25 | 120 | 60 | 150 | 0.1 | 4B | 20.00 |
| 5 | 25 | 180 | 120 | 50 | 0.2 | 4B | 13.98 |
| 6 | 25 | 240 | 30 | 150 | 0.3 | 3B | 10.46 |
| 7 | 50 | 120 | 120 | 150 | 0.3 | 3B | 10.46 |
| 8 | 50 | 180 | 30 | 150 | 0.4 | 2B | 7.96 |
| 9 | 50 | 240 | 60 | 50 | 0.4 | 2B | 7.96 |

Notes*:
The adhesion grades are evaluated by ASTM D3359 for coating adhesion and are divided into six scales including 5B, 4B, 3B, 2B, 1B and 0B which have corresponding definitions as follows:
5B: The coating is not damaged at edges and intersections of the lattice.
4B: The actual damaged area in the lattice is smaller than 5%.
3B: The actual damaged area in the lattice is between 5% and 15%.
2B: The actual damaged area in the lattice is between 15% and 35%.
1B: The actual damaged area in the lattice is between 35% and 65%.
0B: The actual damaged area in the lattice is greater than 65%.

In view of the foregoing, in the signal transmitting element according to the present invention, by the body 1 having a laminated structure formed of a biodegradable material and by the signal transmitting portion STP and the signal sensing portion SSP which respectively senses and transmits the corresponding blood vessel signal, continuous monitoring of the target blood vessel 2 of a patient within a specific period of time after surgery can be achieved. Furthermore, after the specific period of time, the signal transmitting element according to the present invention can be degraded in the patient's body without additional surgery for removal, avoiding the risks of the additional surgery. Furthermore, by providing the plurality of protruding structures 3 in the locations corresponding to the target blood vessel to be monitored, particularly through contact of the plurality of protruding structures 3 and the target blood vessel, the signal strength of the blood flow information sensed by the signal sensing portion SSP can be enhanced. Furthermore, by providing the plurality of metal structures 6 whose signal transmitting sections 6a have the same working frequency, the signal transmission strength of the working frequency can be enhanced, or by providing the plurality of metal structures 6 whose signal transmitting sections 6a have different working frequencies, the breadth of the signal transmission frequency can be increased. Furthermore, by electro-plating the magnesium wires constituting the metal structures 6, the reflection loss can be reduced to provide a low impedance, thereby increasing the radiation efficiency and reducing the impedance loss. Furthermore, by the specific plating condition provided by the present invention, a signal transmitting section 6a with a low resistance and a high adhesion can be obtained.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims.

What is claimed is:

1. A signal transmitting element comprising:
a body made of a biodegradable material and including:
a signal sensing portion including a specific structure configured to sense a blood flow information of a blood vessel surrounded and contacted by the body, thereby generating a blood vessel signal; and
a signal transmitting portion coupled with the signal sensing portion for receiving the blood vessel signal and including a specific structure configured to convert the blood vessel signal into a transmission signal.

2. The signal transmitting element as claimed in claim 1, wherein the body is in a form of a strip and includes a head portion and an extension portion extending outwards from the head portion, wherein the extending portion completely or partially surrounds the blood vessel, wherein the signal transmitting portion is disposed in the head portion, and wherein the signal sensing portion is disposed in the extension portion.

3. The signal transmitting element as claimed in claim 2, wherein the extension portion includes a plurality of protruding structures protruding from a surface thereof, such that when the extension portion surrounds the blood vessel, all or a portion of the plurality of protruding structures contacts with the blood vessel.

4. The signal transmitting element as claimed in claim 2, wherein the body includes at least one metal structure, wherein each of the at least one metal structure is made of a biodegradable metal, wherein each of the at least one metal structure includes a signal transmitting section and a signal sensing section, wherein the signal transmitting section is located in the head portion of the body to form the signal transmitting portion, wherein the signal sensing section is located in the extension portion of the body to form the signal sensing portion, wherein the signal transmitting section has a working frequency used for transmitting the blood flow information sensed by the signal sensing section.

5. The signal transmitting element as claimed in claim 4, wherein the at least one metal structure includes a plurality of metal structures, wherein the working frequency of one of the plurality of metal structures is identical to that of another of the plurality of metal structures.

6. The signal transmitting element as claimed in claim 4, wherein the at least one metal structure includes a plurality of metal structures, wherein the working frequency of one of the plurality of metal structures is different from that of another of the plurality of metal structures.

7. The signal transmitting element as claimed in claim 4, wherein the working frequency is between 350 MHz and 450 MHz.

8. The signal transmitting element as claimed in claim 4, wherein the working frequency is between 401 MHz and 406 MHz.

9. The signal transmitting element as claimed in claim 4, wherein a material of each of the at least one metal structure is at least selected from magnesium or magnesium alloy.

10. The signal transmitting element as claimed in claim 9, further comprising a gold-plated layer coated on a surface of at least one of the at least one metal structure.

11. The signal transmitting element as claimed in claim 10, wherein the gold-plated layer has a thickness between 0.01 μm and 10 μm.

12. The signal transmitting element as claimed in claim 10, wherein the gold-plated layer is formed under a condition including a plating temperature of 15° C.-35° C., a pretreatment time of 60-150 seconds, a plating time of 45-75 seconds, and a stirring speed of 100-300 rpm.

13. The signal transmitting element as claimed in claim 11, wherein the gold-plated layer is formed under a condition including a plating temperature of 25° C., a pretreatment time of 120 seconds, a plating time of 60 seconds, and a stirring speed of 150 rpm.

14. The signal transmitting element as claimed in claim 4, wherein the body includes a first protective layer and a second protective layer, wherein the at least one metal layer is enveloped between the first protective layer and the second protective layer, wherein the plurality of protruding structures is made of a material including at least polycaprolactone, and wherein the first protective layer and the second protective layer are made of a material selected from at least one of polyhydroxybutyrate and polyhydroxyvalerate.

15. The signal transmitting element as claimed in claim 4, wherein the at least one metal structure includes a plurality of metal structures, wherein each two adjacent metal structures have an insulating layer disposed therebetween, and wherein the insulating layer is made of a material including at least poly-L-lactic acid.

16. The signal transmitting element as claimed in claim 2, wherein the head portion has a length between 5 mm and 35 mm, a width between 5 mm and 35 mm, and a thickness between 50 μm and 350 μm, and wherein the extension portion has a width between 2 mm and 15 mm and a thickness between 50 μm and 350 μm.
